(19) United States
(12) Patent Application Publication
(10) Pub. No.: US 2002/0169471 A1
(43) Pub. Date: Nov. 14, 2002

(54) Insertion devices and method of use

(75) Inventor: Arthur E. Ferdinand, San Jose, CA (US)

Correspondence Address: RYAN KROMHOLZ & MANION, S.C., Post Office Box 26618, Milwaukee, WI, 53226, US

(73) Assignee: KYPHON INC.

(21) Appl. No.: 10/123,869

(22) Filed: Apr. 16, 2002

Related U.S. Application Data
(60) Provisional application No. 60/283,990, filed on Apr. 16, 2001.

(51) Int. Cl. A61B 17/34

(52) U.S. Cl. 606/185

(57) ABSTRACT

This invention relates to a surgical access instrument having a distal tip which facilitates introduction of the instrument into and through hard or dense tissue, but which facilitates removal of some or all of the instrument through the application of minimal withdrawal forces.

### RELATED APPLICATION

This application claims the benefit of U. S. provisional patent application Serial No. 60/283,990 filed Apr. 16, 2001.

### FIELD OF THE INVENTION

This invention generally relates to hand-held tools and instruments and to procedures that deploy these instruments through tissue to access interior regions of the body.

### BACKGROUND OF THE INVENTION

There are many different types and styles of hand-held surgical instruments that physicians use to gain access into interior body regions. These instruments are intended to penetrate tissue by the application of pushing forces, twisting forces, or both in combination.

Often, a single surgical procedure will require the physician to employ different surgical instruments, each possessing a different size, shape and function. The procedure will typically require the physician to deploy these instruments in both soft and hard tissue to meet the diagnostic or therapeutic objectives of the procedure. The physician will often need an enhanced mechanical advantage to advance an instrument through tissue, particularly through dense or hard tissue such as bone. Often, surgical hammers or mallets are often utilized to advance these instruments through such hard or dense tissues.

Where surgery is conducted is the proximity of vital areas of the body, such as near the brain, other nerves, major veins or arteries, it is often preferred to make an initial approach using a very small diameter needle, such as a spinal needle. A stylet or guide wire may then be positioned to establish a safe access path to the surgical site, along which any number of larger surgical tools can be advanced. Such larger tools are typically cannulated so that the stylet or guide wire passes through a lumen in at least a portion of the larger surgical tools, desirably guiding the tool to the surgical site.

Once access has been achieved and/or the surgical procedure has been completed, the surgical instruments are generally removed from the patient. Where the surgical tools were difficult to insert through dense or hard tissue, however, they will often be difficult to remove from this tissue as well. Various surgical instruments have been created to facilitate removal of “stuck” tools, some similar to claw-hammers or crowbars, which desirably give a physician a mechanical advantage, thereby increasing the physician's ability to withdraw the tool. Similarly, reverse-impacting devices or “slap-hammers” have been developed which use the momentum developed by a moving mass to increase the physician's ability to pull on surgical tools with increasing force. While these devices magnify the practitioner's strength, allowing surgical tools to be removed from such harder tissues, they do not address the underlying problem of reducing the tendency for such tissues to retain such instruments in the first place.

### SUMMARY OF THE INVENTION

The present invention provides a surgical access instrument having a distal tip which facilitates introduction of the instrument into and through dense tissue, but which facilitates removal of some or all of the instrument through the application of minimal withdrawal forces.

One aspect of the invention provides a tool comprising a first functional instrument and a second functional instrument. The first and second functional instruments engage to form a composite tool. The composite tool has a distal tip particularly suited for advancement and retraction from dense or hard tissue such as bone. In one embodiment, the distal tip is cannulated to allow the tool to be advanced along a stylet or guide wire into a targeted tissue region. In another embodiment, the distal tip is solid to allow the tool to cut through tissue.

In a general embodiment of the present invention, the distal tip of the composite tool comprises a plurality of facet faces, which desirably present a non-continuous and/or non-uniform surface to the dense tissue through which the tool passes. During introduction of the tool, directly or along a stylet or guide wire, these faces facilitate separation of soft and hard tissue planes, desirably minimizing trauma to such tissues. In addition, as the instrument is withdrawn from the hard or dense tissue, the tendency for the distal tip to “stick” in the dense or hard tissue is minimized, thereby reducing the amount of force required to remove the tool from such tissues. By presenting a non-continuous surface to the dense tissue, the tip significantly reduces the amount of frictional and/or retention forces experienced by the tool, and significantly reduces the size and/or effect of the “locking zone” on the distal tip of the tool. The present invention further minimizes the surface area against which retention forces may act.

Other objects, advantages and embodiments of the invention are set forth in part in the description that follows, and in part, will be obvious from this description, or may be learned from the practice of the invention.

### BRIEF DESCRIPTION OF THE DRAWINGS

FIG. 1 is a perspective view of one embodiment of a composite tool constructed in accordance with the teachings of the present invention, separated into its component parts;

FIG. 2 is a perspective view of the composite tool of FIG. 1;

FIG. 3 is a front perspective view of the composite tool of FIG. 1;

FIG. 4 is a side view of one embodiment of a trocar constructed in accordance with the teachings of the present invention;

FIG. 5 is an end view of the trocar of FIG. 4, taken along line 5-5;

FIG. 6 is a cross-sectional view of the trocar of FIGS. 4 and 5, taken along line 6-6;

FIG. 7 is a cross-sectional view of the trocar of FIGS. 4 and 5, taken along line 7-7.

FIG. 8 is a side view of one embodiment of a trocar constructed in accordance with the teachings of the present invention.

FIG. 9 is an end view of the trocar of FIG. 8.

### DETAILED DESCRIPTION OF PREFERRED EMBODIMENTS

The present invention overcomes the problems and disadvantages associated with current strategies and designs in insertion devices for use in accessing hard and/or dense tissues. In particular, the present invention describes insertion devices which may be used in treating various locations within human and/or animal bodies, such as the methods and instruments described in U.S. Pat. Nos. 4,969,888, 5,108,404, 5,827,289, 5,972,015, 6,048,346 and 6,066,154, each of which are incorporated herein by reference.

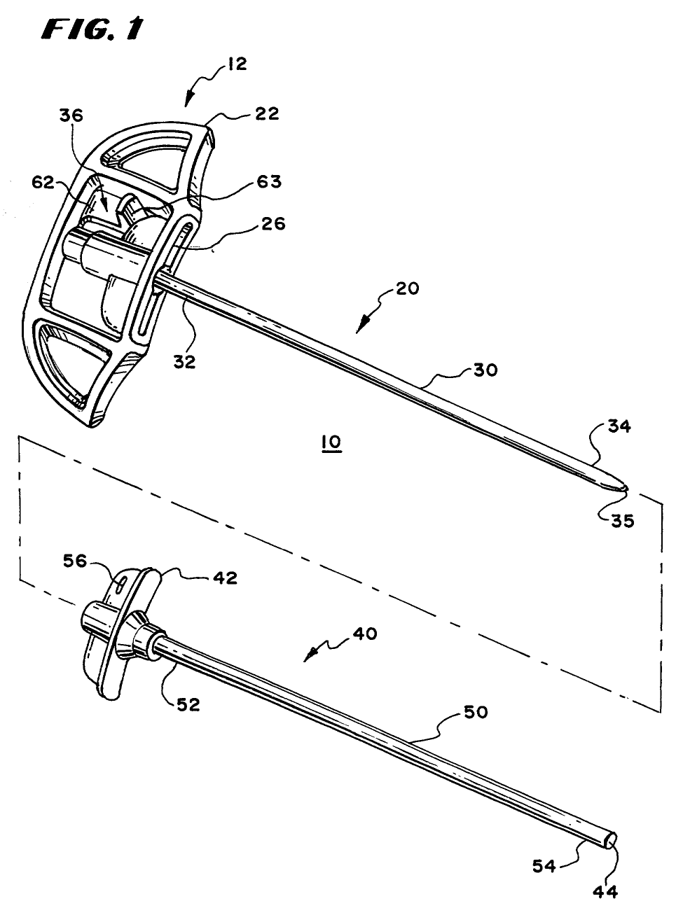

FIG. 1 shows a composite instrument 10 for penetrating tissue. The composite instrument 10 includes a first functional instrument 20 and a second functional instrument 40, and a composite handle 12 comprising a first handle 22 and a second handle 42. The composite handle 12 aids a physician in manipulating the composite instrument 10, but a physician can also desirably use the first handle 22 to independently manipulate the first instrument 20 or the second handle 42 to independently manipulate the second instrument 40 during use.

The number and type of instruments 20 and 40 can vary. FIG. 1 shows two representative instruments 20 and 40, each having a different size and function. In one embodiment, the first functional instrument 20 is a trocar instrument, and the second functional instrument 40 is a cannula instrument. The first instrument 20 functions as a trocar instrument to penetrate tissue. A trocar has a proximal end 32 and a distal end 34. The distal end 34 is tapered to present a penetrating surface 35. In use, the penetrating surface 35 is intended to penetrate soft tissue and/or hard, dense tissue in response to pushing and/or twisting forces applied by the physician at the first handle 22, or the composite handle 12.

The first handle 22 is coupled to the trocar 30 at the proximal end of the trocar 32. If desired, the proximal end of the trocar 30 could be formed in a T-shape (not shown), with the first handle 22 being molded around the T-shaped end. This arrangement would significantly increase the mechanical strength of the bond between the handle 22 and the trocar 30, and allows significant longitudinal and torsional forces to be transmitted from the handle 22 to the trocar 30 without bond failure. Alternatively, with or without a T-shaped end, the proximal end 32 of the trocar 30 can be scored (not shown) to increase the mechanical strength of the bond between the trocar 30 and the handle 22, or various bonding adhesives could be used, with varying results.

In an alternate embodiment, the trocar 30 includes an interior lumen (not shown), which passes through the handle 22 and the body of the trocar 30. The interior lumen accommodates the passage of a stylet and/or conventional spinal needle assembly, to guide the deployment of the first instrument 20, by itself or nested with the second instrument 40, through soft tissue to a targeted hard and/or dense tissue such as bone.

The second instrument 40 functions as a cannula instrument or guide sheath, and includes a cannula 50. The cannula 50 of the second instrument 40 is desirably somewhat larger in diameter than and not as long as the trocar 30 of the first instrument 20. The second instrument 40 includes an interior lumen 44 that extends through the instrument from its distal end 54 to its proximal end 52. The interior lumen 44 is sized to accept the trocar 30. The size of the lumen 44 desirably allows each instrument to slide and/or rotate relative to the other when the handles are not engaged.

The distal end 54 of the second instrument 40 presents an end surface 60. In use, the end surface 60 of the second instrument 40 desirably presents a low-profile surface, which can penetrate soft tissue surrounding the first instrument 20 in response to pushing and/or twisting forces applied at the composite handle 12 or the second handle 42.

The proximal end 52 is coupled with the second handle 42. If desired, the proximal end 52 of the cannula can be flared and/or notched (not shown), with the second handle 42 molded around the proximal end 52. The flared and/or notched proximal end can increase the mechanical strength of the bond between the cannula 50 and the second handle 42, allowing significant longitudinal and torsional forces to be transmitted between the second handle 42 and the cannula 50 without bond failure. As with the trocar 30, however, alternative bonding methods such as scoring of the cannula 50 and/or the use of various adhesives could be employed, with varying results.

The first handle 22 and the second handle 42 are designed to comfortably accommodate a hand, to desirably interlock to form a composite handle 12 that resists relative movement between the first and second instruments during introduction into and/or removal from hard or dense tissue. The first handle 22 desirably includes a receiving channel 26 with a latch mechanism 36 that engages a corresponding latch notch 56 on the second handle 42. In one embodiment, the latch mechanism includes a latch finger 63 situated to engage the latch notch of the second handle 42. The latch finger is carried on a hinge 62 in the first handle 22. The hinge 62 is desirably made from resilient plastic material and possesses plastic memory, forming a flexible hinge.

The latch finger 60 is cantilevered on the hinge 62 for pivoting movement within the first handle 22. The plastic memory of the hinge 262 normally biases the finger 60 toward a normal position, in which the finger will rest within the notch 56, providing that the two parts are in alignment. The latch finger 60 can be displaced out of its normal alignment in response to an applied force from the practitioner desiring to separate the two instruments.

Once the composite tool is located within a desired position in the hard or dense tissue, the first instrument 20 can be removed from the second instrument 40, such that the interior lumen 44 of the second instrument 40 provides an access passageway into and/or through the hard or dense tissue. Desirably, the practitioner will depress the latch finger 60, which disengages the first handle from the second handle, and then the practitioner can withdraw the trocar 30 from the interior lumen 44.

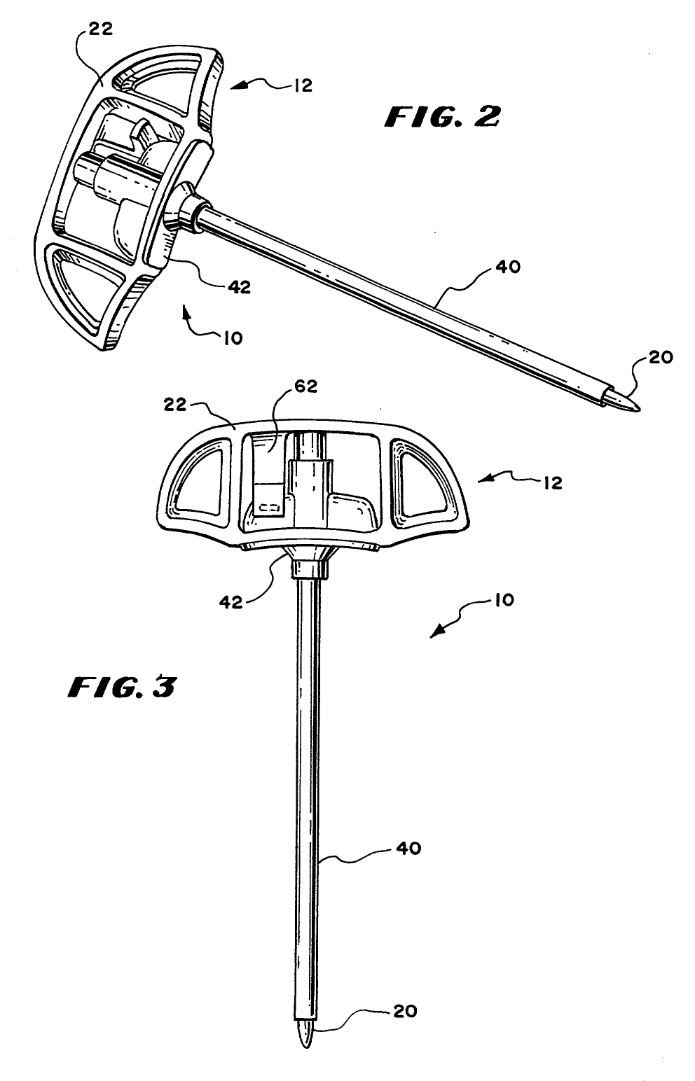

Prior to such removal, the distal tip of the trocar 30 typically extends out of the distal tip of the cannula 50, and is generally in contact with the dense or hard tissue. This tissue, which contacts the trocar 30, will generally resist withdrawal of the trocar 30 into the interior lumen 44. This resistance is created by various factors, one of which can be frictional forces induced by the tissue on the shaft/distal tip of the trocar. The surfaces of a smooth, rounded distal tip (such as shown in FIGS. 1 through 3) will often be held in a “self-locking” region of the hard or dense tissue, at which point the force required to withdraw the tip tends towards a maximum value. To withdraw the trocar 30 from this region of tissue, the practitioner will often have to exert considerable force, sometimes on the order of fifty or one-hundred (50 or 100) or more pounds of force. Moreover, because rotation of the cannula 50 is often undesirable at this point, and the first and second handles typically inhibit independent rotation during initial withdrawal of the trocar 30, rotation of the trocar 30 is generally precluded, possibly rendering the required pullout forces to even greater amounts.

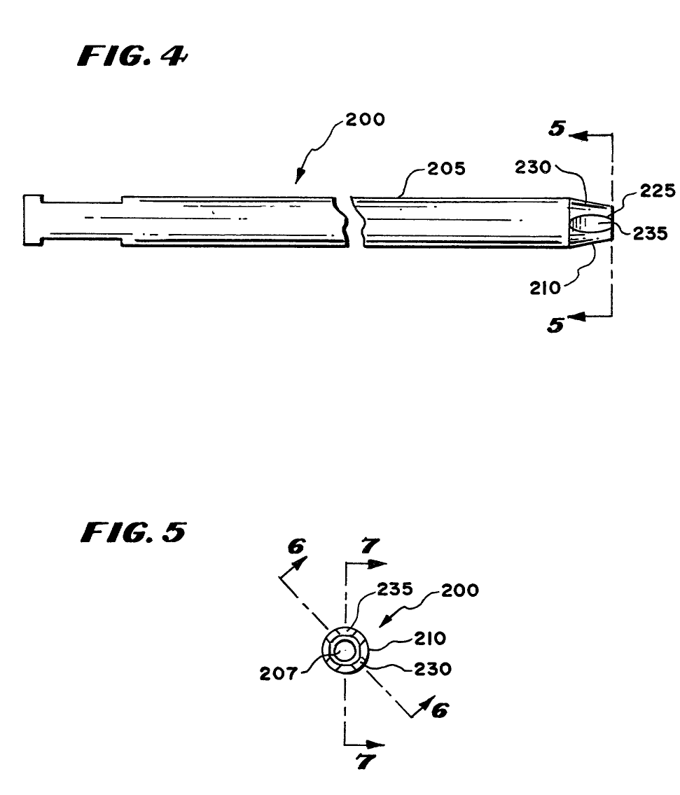

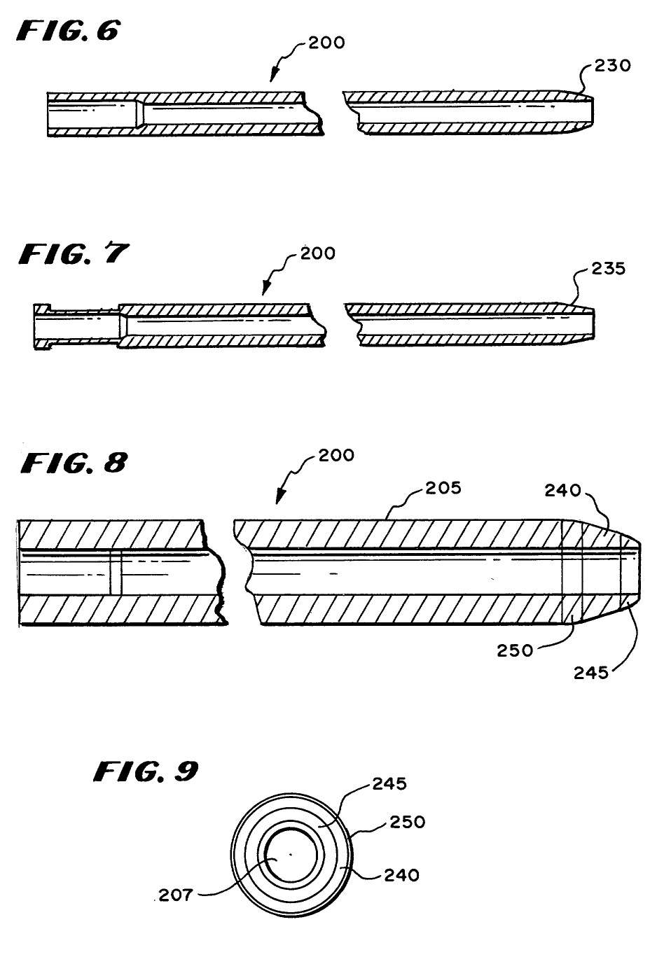

The present invention significantly reduces the pullout forces necessary to remove a trocar from hard and/or dense tissue. FIG. 4 depicts one embodiment of a trocar 200 which incorporates a distal tip 210 constructed in accordance with the teachings of the present invention. Because many of the features of this trocar are similar to those previously described, like reference numerals will be used to denote similar components.

The trocar 200 includes a shaft 205 and a distal tip 210. A lumen 207 desirably extends through the central axis of the trocar 200. The distal tip 210 incorporates a plurality of angled facets 225 which desirably provide a smooth transition from the distal tip 210 of the trocar 200 to the distal tip of the cannula 50 and, during advancement of the composite tool through soft and/or hard tissues, facilitate separation of tissue planes to minimize tissue trauma and permit advancement of the cannula through tissues. The facets 225 comprise rounded sections 230 and flat sections 235, which in the disclosed embodiment are distributed symmetrically about the distal tip 210. Of course, if desired these sections 230 and 235 could be distributed in various alternate arrangements, including non-symmetrically about the distal tip 210 of the instrument. In at least one alternate embodiment, a small section of the shaft 205 may also extend from the distal tip of the cannula.

In this embodiment, as the trocar 200 is withdrawn from the cannula 50, the hard or dense tissue will typically oppose removal of the instrument. Generally, forces opposing removal can comprise the frictional forces between the tissue and shaft 205 as well as frictional forces between the tissue and distal tip 210. As the trocar 200 is first withdrawn, the distal tip 210 of the shaft 205 may be located within a “self locking” region of the tissue, in which the forces attempting to retain the tip tend towards a maximum value. This “self locking” region is generally dependent, at least in part, upon the geometry of the distal tip. By incorporating multiple facet faces, however, the non-uniform profile of the distal tip 210 desirably alters the size and/or effect of the “self locking” region, desirably reducing the magnitude of the force opposing withdrawal of the instrument. Moreover, in another embodiment, the trocar 200 is desirably sized such that, when mated with the cannula 50, only the distal tip 210 of the trocar 200 extends from the distal tip 60 of the cannula 50. Accordingly, during withdrawal of the trocar 200 from the cannula 50, only the distal tip 210 of the trocar 200 encounters resistance from the hard or dense tissue, further reducing overall withdrawal forces. The cannula 50 may also be faceted to ease withdrawal of the tool.

Alternatively, as shown in FIGS. 8 and 9, the trocar 200 includes a shaft 205 and a distal tip 210. A lumen 207 desirably extends through the central axis of the trocar 200. The distal tip 210 incorporates a single facet 240 which encircles the trocar 200. The facet 240 is flat, and the sections 245 and 250 are rounded. The single facet face 240 desirably disrupts the size and/or effect of the “self locking” region, desirably reducing the magnitude of the force opposing withdrawal of the instrument.

The instruments described herein may be comprised of a generally rigid material common in medical device applications, including, but not limited to, plastics, metals, ceramics or composite materials. In one embodiment, the instruments are comprised of stainless steel. While the disclosed devices and methods are more specifically described in the context of the treatment of human vertebrae, other human or animal bone types can be treated in the same or equivalent fashion. By way of example, and not by limitation, the present systems and methods could be used in any bone having bone marrow therein, including the radius, the humerus, the vertebrae, the femur, the tibia or the calcaneous.

Other embodiments and uses of the invention will be apparent to those skilled in the art from consideration of the specification and practice of the invention disclosed herein. All documents referenced herein are specifically and entirely incorporated by reference. The specification and examples should be considered exemplary only, with the true scope and spirit of the invention being indicated by the following claims. As will be easily understood by those of ordinary skill in the art, variations and modifications of each of the disclosed embodiments, including combinations thereof, can be easily made within the scope of this invention as defined by the following claims.

What is claimed is:

1. A surgical instrument for accessing hard or dense tissue, the instrument comprising
a trocar having a proximal end and a distal end;
a lumen extending through the interior of the trocar,
the distal end including a distal tip;
the distal tip comprising a plurality of facet faces.

2. The instrument of claim 1, wherein the plurality of facet faces comprises at least one rounded facet face and at least one flat facet face.

3. The instrument of claim 1, wherein the facet faces are non-continuous.

4. The instrument of claim 1, wherein the facet face is continuous.

5. The instrument of claim 1, wherein the facet faces are non-uniform.

6. A surgical instrument for accessing hard or dense tissue, the instrument comprising
a trocar having a proximal end and a distal end;
the distal end including a distal tip;
the distal tip comprising a plurality of facet faces.

7. The instrument of claim 1, wherein the facet faces are non-continuous.

8. The instrument of claim 1, wherein the facet face is continuous.

9. The instrument of claim 1, wherein the facet faces are non-uniform.
